(19) United States
(12) Patent Application Publication
(10) Pub. No.: US 2010/0234713 A1
(43) Pub. Date: Sep. 16, 2010

(54) SILVER-SILVER CHLORIDE NEEDLE ELECTRODE SYSTEM

(76) Inventor: DAVID A. SHERATON, SR., Aliso Viejo, CA (US)

Correspondence Address: CHARLES C.H. WU, 98 DISCOVERY, IRVINE, CA, 92618-3105, US

(21) Appl. No.: 12/401,769

(22) Filed: Mar. 11, 2009

(51) Int. Cl. A61B 5/04

(52) U.S. Cl. 600/373

(57) ABSTRACT

A needle electrode system for acquiring biopotential signals from a person's body. The needle electrode system includes at least one silver-silver chloride needle electrode that is coupled to a distal end of a first wire, at least one cutaneous electrode connector coupled to a distal end of a second wire and at least one connector attached to at least one proximal end of the first wire and the second wire. The connector is coupled with a monitoring device. The first wire and the second wire are made of carbon fiber. The non-polarizable needle electrode that is made out of or coated with silver-silver chloride has a protective safety cover. The needle electrode system senses and sends biopotential signals to the monitoring device in preference to cutaneous electrode on the person's body by inserting the needle electrode subcutaneously into the person's body.

### BACKGROUND OF THE INVENTION

1. Technical Field of the Invention

The present invention relates in general to a needle electrode system having a needle electrode made out of or coated with silver-silver chloride. More specifically, the present invention relates to a needle electrode system for acquiring biopotential signals from a person's body.

2. Description of the Related Art

The advancement of needle electrodes has been revolutionizing the modern medical diagnosis and treatment over a long time. Needle electrodes are invasive bio-electrodes that are inserted into a person's body for acquiring biopotential signals. Needle electrodes sense and collect electrophysiological information such as ECG, EEG, EMG and intracellular potentials. These devices transform biochemical phenomena into electrical currents. The presence, shape, and size of a waveform produced on an electronic monitoring device provide information about the ability of the person's body to respond to a nervous stimulation.

Several biopotential monitoring systems utilize surface electrodes for sensing and sending biopotential signals. Surface electrodes are placed on a person's body for gathering biopotential signals. A common problem associated with surface electrodes is the susceptibility of interference to the acquired signal from adjacent electrodes. Moreover, the signals acquired through surface electrodes are prone to crosstalk.

One type of needle electrode system that can be inserted into a person's body for sensing biopotential signals overcomes the drawbacks of the surface electrodes. These needle electrodes are made with stainless steel. However, these needle electrodes are polarizable and are susceptible to charge build up, when monitoring low frequency signals. As a result, the impedance remains high thereby rendering the system incapable to sense and send accurate biopotential signals to a monitoring device.

A type of needle electrode system for recording facial EMG utilizes a pair of needle electrodes. Such a mechanism causes inconvenience to a patient while monitoring EMG signals. Furthermore, such a mechanism requires detachable parts that are complex to manufacture.

Another needle electrode assembly includes a holder that releaseably holds needle electrodes of various sizes, shapes, and lengths. The needle electrode-holder is operated by placing the needle electrode holder in a hand and depressing the base of the holder to release or insert a needle electrode. The needle electrode is inserted into the tip of the needle electrode holder while depressing the base of the needle electrode holder. However, the assembly requires an internal engagement mechanism of the needle electrode holder and a gripping assembly located within the body to engage and hold the needle in place. In addition, the needle electrode holder is to be held in a person's hand in a recommended position causing a relatively complex procedure to acquire the biopotential signals from a person's body.

Many attempts are made to manufacture simple needle assembly with lesser detachable parts. Such an attempt introduced a needle assembly with a non detachable holder. This approach is difficult and expensive to manufacture.

Hence, it can be seen that there is a need for a needle electrode system that would be capable of sensing and sending biopotential signals from very small muscles without interference and cross talk. Such a device would be non-polarizable and capable of dissipating charge built up when monitoring low frequency signals. Further, the device would reduce the impedance offered by the person's body thereby senses and sends accurate biopotential signals. Moreover, the needed device would be economical, and easy to manufacture.

### SUMMARY OF THE INVENTION

To minimize the limitations found in the prior art, and to minimize other limitations that will be apparent upon the reading of the specifications, the present invention provides a needle electrode system for acquiring biopotential signals from a person's body. The needle electrode system comprises at least one silver-silver chloride needle electrode that is coupled to a distal end of a first wire, at least one cutaneous electrode connector coupled to a distal end of a second wire and at least one connector attached to at least one proximal end of the first wire and the second wire. The connector is coupled with a monitoring device. The needle electrode system senses and sends biopotential signals to the monitoring device in preference to the cutaneous electrode on the person's body by inserting the silver-silver chloride needle electrode subcutaneously into the person's body.

In another aspect of the present invention, a medical tape affixes a cutaneous electrode connector on a person's body. The process of acquiring biopotential signals from a person's body is initiated by coupling at least one connector with a monitoring device where the connector is attached to at least one proximal end of a first wire and a second wire. A cutaneous electrode connector is placed on a surface of the person's body where the cutaneous electrode connector is coupled to a distal end of the second wire and may or may not be connected to a cutaneous electrode. A silver-silver chloride needle electrode is inserted subcutaneously into the person's body if needed where the silver-silver chloride needle electrode is coupled to a distal end of the first wire.

One objective of the invention is to provide a needle electrode system with a needle electrode coated with or made of silver-silver chloride.

Another objective of the invention is to provide a needle electrode system with an electrically conductive connector coupling at least one electrically conductive first wire and second wire.

A third objective of the invention is to provide a needle electrode system with a first wire and a second wire that may be a carbon fiber in place of a metal.

Yet another objective of the invention is to provide a needle electrode system with a non-polarizable needle electrode having a protective safety cover.

Still another objective of the invention is to provide a needle electrode system with a medical tape that provides further adhesion of a cutaneous electrode connector to a surface of a person's body.

It is also the objective of the invention to provide a needle electrode system with a cutaneous electrode connector as a means to hold to a person's body.

These and other advantages and features of the present invention are described with specificity so as to make the present invention understandable to one of ordinary skill in the art.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention, thus the drawings are generalized in form in the interest of clarity and conciseness.

FIG. 1 is a schematic view of the present invention showing the needle electrode system;

FIG. 2 is an enlarged schematic view of the present invention showing the silver-silver chloride needle electrode with the protective safety cover;

FIG. 3A is a schematic view of the present invention showing the needle electrode system ready to be used for acquiring biopotential signals, the cutaneous electrode connector being affixed with the medical tape;

FIG. 3B is a schematic top plan view of the medical tape; and

FIG. 4 is a flow diagram showing the method of use of the needle electrode system for acquiring biopotential signals from the person's body.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

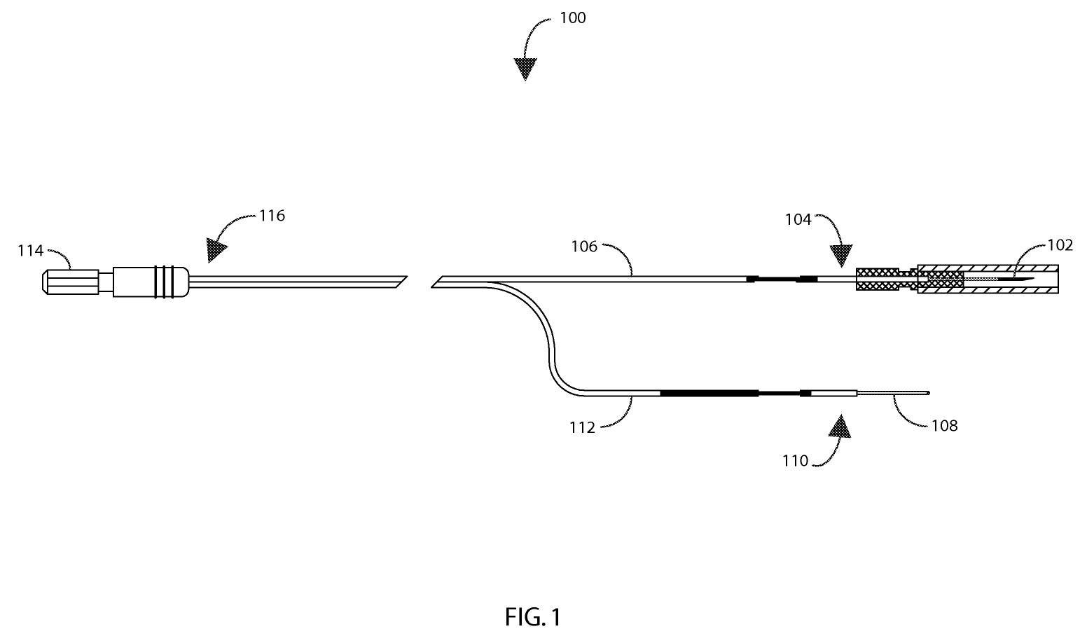

FIG. 1 shows a schematic view of the present invention showing a needle electrode system 100 in accordance with the present invention.

The needle electrode system 100 acquires biopotential signals (not shown) from a person's body (not shown). The system 100 comprises at least one silver-silver chloride needle electrode 102 that is coupled to a distal end 104 of a first wire 106, at least one cutaneous electrode connector 108 coupled to a distal end 110 of a second wire 112 and at least one connector 114 attached to at least one proximal end 116 of the first wire 106 and the second wire 112. The connector 114 is coupled with a monitoring device (not shown). The needle electrode system 100 senses and sends biopotential signals (not shown) to the monitoring device (not shown) in preference to the cutaneous electrode connector 108 on the person's body (not shown) by inserting the silver-silver chloride needle electrode 102 subcutaneously into the person's body (not shown).

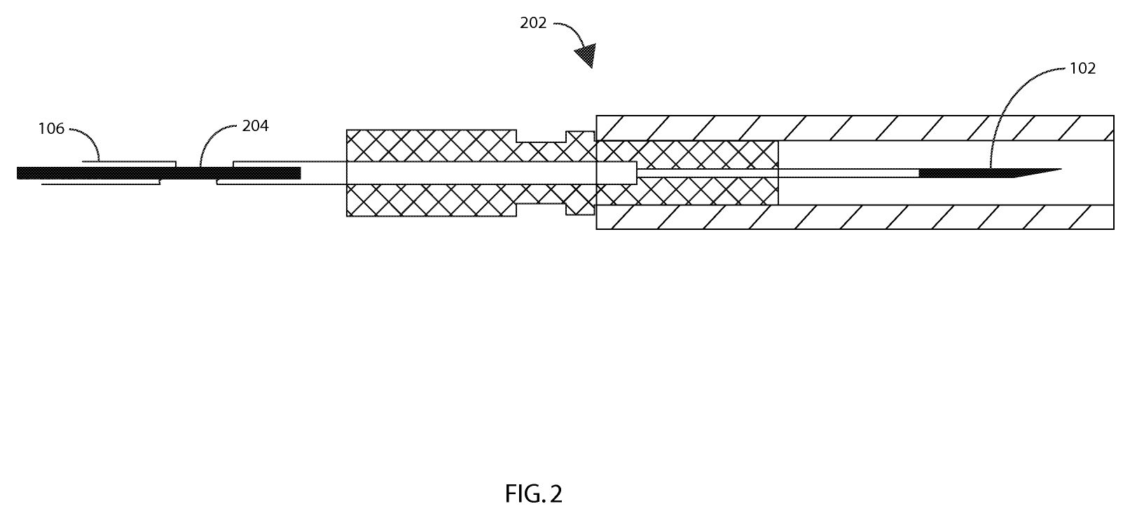

FIG. 2 is an enlarged schematic view of the silver-silver chloride needle electrode 102 with a protective safety cover 202. The needle electrode 102 may be made out of or coated silver-silver chloride. The silver-silver chloride needle electrode 102 includes at least one protective safety cover 202 encasing thereof. The first wire 106 and the second wire 112 are electrically conductive. The first wire 106 and the second wire 112 are made of carbon fiber 204 in place of a metal.

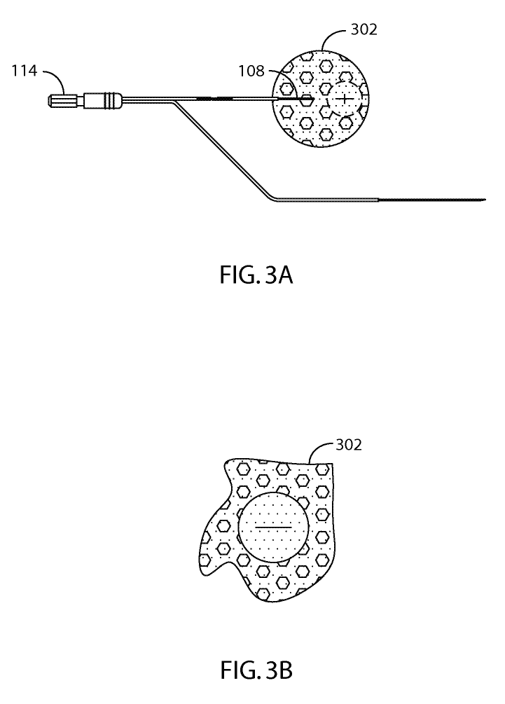

FIG. 3A is a schematic view of the present invention showing the needle electrode system 100 ready to be used for acquiring biopotential signals (not shown). The cutaneous electrode connector 108 via the cutaneous electrode 304 is the means to hold the needle electrode system 100 to the person's body (not shown). At least one medical tape 302 provides further adhesion of the cutaneous electrode connector 108 to the surface of the person's body (not shown). The connector 114 attached to at least one proximal end 116 of the first wire 106 and the second wire 112 with the monitoring device (not shown) is electrically conductive. The needle electrode 102 having a low impedance and a stable offset voltage facilitates dissipation of charge build up in the needle electrode 102 when monitoring low frequency signals from the person's body (not shown) thereby records accurate biopotential signals (not shown). The silver-silver chloride needle electrode 102 is non-polarizable.

FIG. 3B is a schematic view of a medical tape 302. The medical tape 302 affixes the cutaneous electrode connector 108 on the person's body (not shown). The silver-silver chloride needle electrode 102 is inserted subcutaneously into the person's body (not shown). The connector 114 attached to at least one proximal end 116 of the first wire 106 and the second wire 112 with the monitoring device (not shown) transmits biopotential signals (not shown).

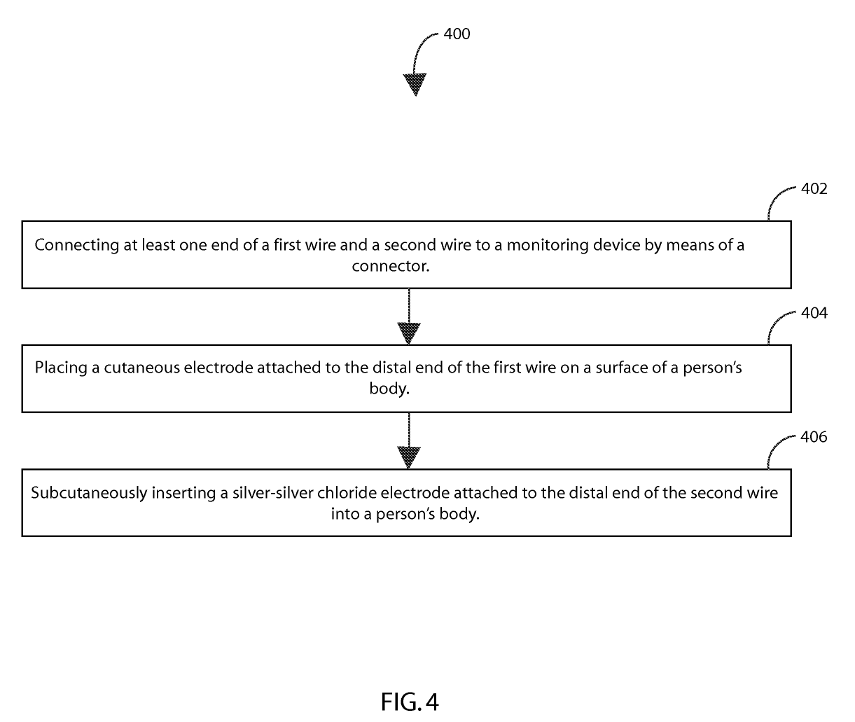

FIG. 4 is a flow diagram 400 showing a method of use of a needle electrode system 100 for acquiring biopotential signals (not shown) from a person's body (not shown). In step 402, at least one connector 114 is coupled with a monitoring device (not shown) where the connector 114 is attached to at least one proximal end 116 of a first wire 106 and a second wire 112. In step 404, a cutaneous electrode connector 108 is placed on a surface of the person's body (not shown) where the cutaneous electrode connector 108 is coupled to a distal end 110 of the second wire 112. In step 406, a silver-silver chloride needle electrode 102 is inserted subcutaneously into the person's body (not shown) if needed where the silver-silver chloride needle electrode 102 is coupled to a distal end 104 of the first wire 106.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. A needle electrode system for acquiring biopotential signals from a person's body, said needle electrode system comprising:
at least one silver-silver chloride needle electrode coupled to a distal end of a first wire;
at least one cutaneous electrode connector coupled to a distal end of a second wire; and
at least one connector attached to at least one proximal end of said first wire and second wire, said connector being coupled with a monitoring device;
whereby said needle electrode system senses and sends biopotential signals to said monitoring device in preference to said cutaneous electrode on a surface of said person's body by inserting said silver-silver chloride needle electrode subcutaneously into said person's body.

2. The needle electrode system of claim 1 wherein said needle electrode may be coated with silver-silver chloride.

3. The needle electrode system of claim 1 wherein said needle electrode includes a protective safety cover.

4. The needle electrode system of claim 1 wherein said first wire and second wire are electrically conductive.

5. The needle electrode system of claim 1 wherein said connector coupled with said monitoring device is electrically conductive.

6. The needle electrode system of claim 1 wherein said first wire and second wire may be a carbon fiber in place of a metal.

7. The needle electrode system of claim 1 wherein said cutaneous electrode connector is a means to hold to said person's body via a cutaneous electrode.

8. The needle electrode system of claim 1 wherein said needle electrode is non-polarizable.

9. The needle electrode system of claim 1 wherein said needle electrode having a low impedance and a stable offset voltage facilitates dissipation of charge build up in said needle electrode when monitoring low frequency signals from said person's body thereby records accurate biopotential signals.

10. A method of using a needle electrode system for acquiring biopotential signals from a person's body, said method comprising:
a) coupling at least one connector with a monitoring device, said connector being attached to at least one proximal end of a first wire and a second wire;
b) placing a cutaneous electrode on a surface of said person's body, said cutaneous electrode connector being coupled to a distal end of said second wire; and
c) inserting a silver-silver chloride needle electrode subcutaneously into said person's body if needed, said silver-silver chloride needle electrode being coupled to a distal end of said first wire.

11. The method of using said needle electrode system of claim 10 wherein said needle electrode system senses and sends biopotential signals from said person's body to said monitoring device.

12. The method of using said needle electrode system of claim 10 wherein a medical tape provides further adhesion to said surface of said person's body.
